(19) United States
(12) Patent Application Publication
(10) Pub. No.: US 2016/0159837 A1
(43) Pub. Date: Jun. 9, 2016

(54) MONOPHOSPHITES COMPRISING A BENZOPINACOL

(71) Applicants: Katrin Marie DYBALLA, Recklinghausen (DE); Robert FRANKE, Marl (DE); Dieter HESS, Marl (DE); Dirk FRIDAG, Haltern am See (DE); Frank GEILEN, Haltern am See (DE); Detlef SELENT, Rostock (DE); Armin BÖRNER, Rostock (DE)

(72) Inventors: Katrin Marie DYBALLA, Recklinghausen (DE); Robert FRANKE, Marl (DE); Dieter HESS, Marl (DE); Dirk FRIDAG, Haltern am See (DE); Frank GEILEN, Haltern am See (DE); Detlef SELENT, Rostock (DE); Armin BÖRNER, Rostock (DE)

(73) Assignee: Evonik Degussa GmbH, Essen (DE)

(21) Appl. No.: 14/957,024

(22) Filed: Dec. 2, 2015

(30) Foreign Application Priority Data
Dec. 4, 2014 (EP) 14196195.3

(51) Int. Cl. C07F 9/6574; C07C 45/50; B01J 31/18; C07F 15/00

(52) U.S. Cl. CPC C07F 9/65742; C07F 15/0073; C07C 45/505; B01J 31/185; B01J 2231/321; B01J 2531/822

(57) ABSTRACT

Monophosphites including a benzopinacol structure and metal complexes thereof are provided. The metal complex compositions are useful as hydroformylation catalysts. The metals of the complex include Rh, Ru, Co and Ir. A method of hydroformylation using the metal complex or the metal complex components is also provided.

### CROSS REFERENCE TO RELATED APPLICATIONS

This application claims priority to European Patent Application No. 14196195.3, filed Dec. 4, 2014, the disclosure of which is incorporated herein by reference in its entirety.

### BACKGROUND OF THE INVENTION

1. Field of the Invention

The invention relates to monophosphites comprising a benzopinacol and the use of these compounds as ligands in hydroformylation processes.

2. Discussion of the Background

The reactions between olefin compounds, carbon monoxide and hydrogen in the presence of a catalyst to give the aldehydes comprising one additional carbon atom are known as hydroformylation or oxo synthesis. The catalysts used in these reactions are frequently compounds of the transition metals of group VIII of the Periodic Table of the Elements. Known ligands are, for example, compounds from the classes of the phosphines, phosphites and phosphonites, each with trivalent phosphorus P(III). A good overview of the state of the hydroformylation of olefins can be found in B. CORNILS, W. A. HERRMANN, “Applied Homogeneous Catalysis with Organometallic Compounds”, vol. 1 & 2, VCH, Weinheim, N.Y., 1996 or R. Franke, D. Selent, A. Börner, “Applied Hydroformylation”, Chem. Rev., 2012, DOI:10.1021/cr3001803.

Every catalytically active composition has its specific benefits. According to the feedstock and target product, therefore, different catalytically active compositions are used.

WO 2009/146984 describes bisphosphites having one or two benzopinacol units. On pages 13/14, these are shown as structures Ia, Ib and Ic. In the two structures Ia and Ib, the two outer units are joined by a central unit having a biphenol unit. This biphenol unit has a tert-butyl group directly adjacent to the oxygen atoms that are joined to the phosphorus atom (see also “A New Diphosphite Promoting Highly Regioselective Rhodium-Catalyzed Hydroformylation” by Detlef Selent, Robert Franke, Christoph Kubis, Anke Spannenberg, Wolfgang Baumann, Burkard Kreidler and Armin Börner in Organometallics 2011, 30, 4509-4514).

WO 2012/041846 describes, on page 16, a bisphosphite (structure VIII) of similar structure to the compound Ia from WO 2009/146984.

The disadvantages of bisphosphites include a relatively high level of cost and inconvenience necessary for preparation thereof. It may therefore often be not viable to use such systems in industrial processes. An additional factor is a comparatively low activity in the hydroformylation of relatively long-chain olefins, which has to be compensated for in terms of the reaction by high residence times, which frequently makes an industrial scale process impractical.

### SUMMARY OF THE INVENTION

It was an object of the present invention to provide phosphites which are easier to prepare compared to the known bisphosphites. As another object these phosphites are to have good properties in the hydroformylation of comparatively long-chain olefins. It was also an object to provide phosphites useful in a hydroformylation reaction and which achieve good yields.

These and other objects are achieved by the present invention, the first embodiment of which includes a compound having one of structures (I) to (V):

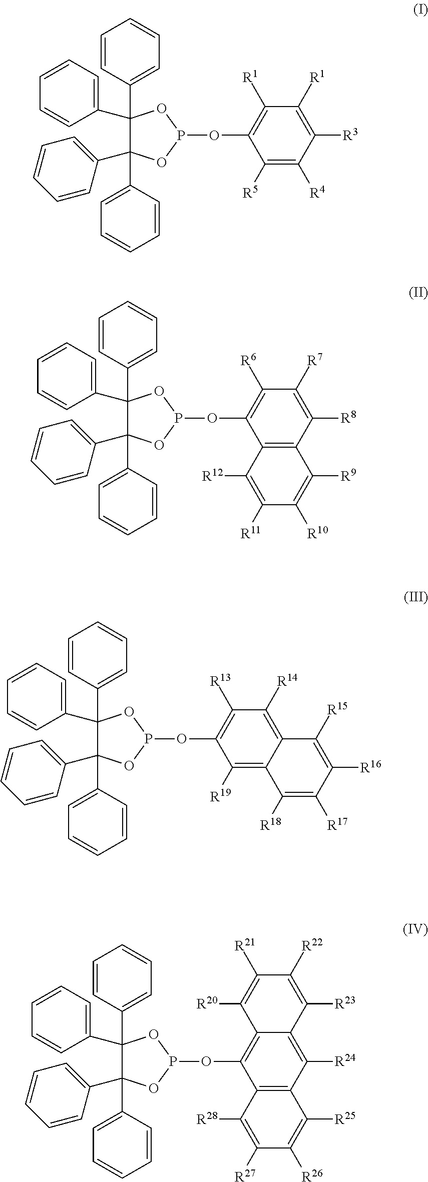

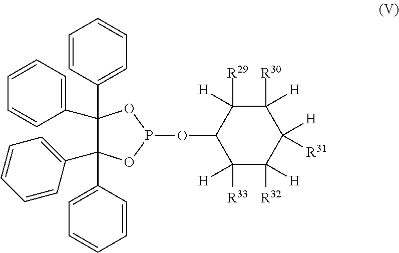

wherein

R¹, R², R³, R⁴, R⁵ are each individually —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂ or —N[(C₁-C₁₂)-alkyl]₂;

R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each individually —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;

R²⁹, R³⁰, R³¹, R³², R³³ are each individually —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;

wherein the alkyl and aryl groups of R¹ to R³⁰ may optionally be substituted,

with the proviso that R¹ and R⁵ are not tert-butyl, at least one of R¹, R², R³, R⁴, R⁵ is not —H, and if one of R¹, R², R³, R⁴, R⁵ is phenyl, at least one of the four remaining R groups is not —H.

In another embodiment the present invention includes a metal ligand complex comprising a monophosphite compound of the first embodiment and a metal atom selected from the group consisting of Rh, Ru, Co and Ir.

In a further embodiment the present invention includes a method for ydroformylation comprising: conducting the hydroformylation reaction in the presence of the metal ligand complex. The method of formulation comprises a) charging an olefin to a reaction device; b) adding a catalyst comprising the metal ligand complex of claim 13 to the reaction device; or adding a catalyst comprising the monophosphite compound of the first embodiment and a substance having a metal atom selected from the group consisting of Rh, Ru, Co and Ir; c) feeding H₂ and CO into the reaction device to the olefin and catalyst to obtain a reaction mixture; and d) heating the reaction mixture to effect conversion of the olefin to an aldehyde.

### DETAILED DESCRIPTION OF THE INVENTION

Any ranges mentioned herein below include all values and subvalues between the lowest and highest limit of the range.

In a first embodiment the present invention provides the monophosphite compounds of structures (I) to (V) described above. In structures (I) to (V):

(C₁-C₁₂)-Alkyl and O—(C₁-C₁₂)-alkyl may each be unsubstituted or substituted by one or more identical or different radicals selected from (C₃-C₁₂)-cycloalkyl, (C₃-C₁₂)-heterocycloalkyl, (C₆-C₂₀)-aryl, fluorine, chlorine, cyano, formyl, acyl and alkoxycarbonyl.

(C₆-C₂₀)-Aryl and —(C₆-C₂₀)-aryl-(C₆-C₂₀)-aryl- may each be unsubstituted or substituted by one or more identical or different radicals selected from: —H, —(C1-C12)-alkyl, —O—(C1-C12)-alkyl, —O—(C6-C20)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂ and —N[(C₁-C₁₂)-alkyl]₂.

In the context of the invention, the expression “—(C₁-C₁₂)-alkyl” encompasses straight-chain and branched alkyl groups. Preferably, these groups are unsubstituted straight-chain or branched —(C₁-C₈)-alkyl groups and most preferably —(C₁-C₆)-alkyl groups. Examples of —(C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The definitions and description relating to the expression “—(C₁-C₁₂)-alkyl” also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted —(C₁-C₁₂)-alkyl groups and substituted —(C₁-C₁₂)-alkoxy groups may have one or more substituents, depending on their chain length. The substituents are preferably each independently selected from —(C₃-C₁₂)-cycloalkyl, —(C₃-C₁₂)-heterocycloalkyl, —(C₆-C₂₀)-aryl, fluorine, chlorine, cyano, formyl, acyl and alkoxycarbonyl.

The expression “—(C₃-C₁₂)-cycloalkyl”, in the context of the present invention, encompasses mono-, bi- or tricyclic hydrocarbyl radicals having 3 to 12, especially 5 to 12, carbon atoms. These include cyclopropyl-, cyclobutyl-, cyclopentyl-, cyclohexyl-, cycloheptyl-, cyclooctyl-, cyclododecyl-, cyclopentadecyl-, norbonyl- and adamantyl. One example of a substituted cycloalkyl may be menthyl.

The expression “—(C₃-C₁₂)-heterocycloalkyl groups”, in the context of the present invention, encompasses nonaromatic saturated or partly unsaturated cycloaliphatic groups having 3 to 12, especially 5 to 12, carbon atoms. The —(C₃-C₁₂)-heterocycloalkyl groups have preferably 3 to 8, more preferably 5 or 6, ring atoms. In the heterocycloalkyl groups, as opposed to the cycloalkyl groups, 1, 2, 3 or 4 of the ring carbon atoms are replaced by heteroatoms or heteroatom-containing groups. The heteroatoms or the heteroatom-containing groups are preferably selected from —O—, —S—, —N—, —N(═O)—, —C(═O)— and —S(═O)—. Examples of —(C₃-C₁₂)-heterocycloalkyl groups are tetrahydrothiophenyl, tetrahydrofuryl, tetrahydropyranyl and dioxanyl.

In the context of the present invention, the expression “—(C₆-C₂₀)-aryl and —(C₆-C₂₀)-aryl-(C₆-C₂₀)-aryl-” encompasses mono- or polycyclic aromatic hydrocarbyl radicals. These have 6 to 20 ring atoms, more preferably 6 to 14 ring atoms, especially 6 to 10 ring atoms. Aryl is preferably —(C₆-C₁₀)-aryl and —(C₆-C₁₀)-aryl-(C₆-C₁₀)-aryl-. Aryl is especially phenyl, naphthyl, indenyl, fluorenyl, anthracenyl, phenanthrenyl, naphthacenyl, chrysenyl, pyrenyl, coronenyl. More particularly, aryl is phenyl, naphthyl and anthracenyl.

Substituted —(C₆-C₂₀)-aryl groups and —(C₆-C₂₀)-aryl-(C₆-C₂₀)-aryl groups may have one or more (e.g. 1, 2, 3, 4 or 5) substituents, depending on the ring size. These substituents are preferably each independently selected from —H, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂, —N[(C₁-C₁₂)-alkyl]₂.

Substituted —(C₆-C₂₀)-aryl groups and —(C₆-C₂₀)-aryl-(C₆-C₂₀)-aryl groups are preferably substituted —(C₆-C₁₀)-aryl groups and —(C₆-C₁₀)-aryl-(C₆-C₁₀)-aryl groups, especially substituted phenyl or substituted naphthyl or substituted anthracenyl. Substituted —(C₆-C₂₀)-aryl groups preferably bear one or more, for example 1, 2, 3, 4 or 5, substituents selected from —(C₁-C₁₂)-alkyl groups, —(C₁-C₁₂)-alkoxy groups.

In one embodiment, R¹, R², R³, R⁴, R⁵ are each independently —H, —(C₁-C₁₂)-alkyl, (C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl or —N[(C₁-C₁₂)-alkyl]₂.

In one embodiment, R⁶, R⁷, R⁸, R⁹, R¹⁰, R¹¹, R¹² are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl or —N[(C₁-C₁₂)-alkyl]₂.

In one embodiment, R¹³, R¹⁴, R¹⁵, R¹⁶, R¹⁷, R¹⁸, R¹⁹ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl or —N[(C₁-C₁₂)-alkyl]₂.

In one embodiment, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl or —N[(C₁-C₁₂)-alkyl]₂.

In one embodiment, R²⁹, R³⁰, R³¹, R³², R³³ are each independently —H, —(C₁-C₁₂)-alkyl, —O—(C₁-C₁₂)-alkyl, —O—(C₆-C₂₀)-aryl, —S-alkyl or —S-aryl.

In one embodiment, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are selected from:

—H, —O—(C₆-C₂₀)-aryl, —(C₆-C₂₀)-aryl, —S-alkyl, —S-aryl, halogen, —CO—(C₁-C₁₂)-alkyl, —CO—(C₆-C₂₀)-aryl, —N[(C₁-C₁₂)-alkyl]₂.

In one embodiment, R¹, R², R³, R⁴, R⁵ are each independently —H, —(C₁-C₁₂)-alkyl, (C₁-C₁₂)-alkyl, —O—(C₆-C₂₀)-aryl, —(C₆-C₂₀)-aryl, —S-alkyl or —S-aryl.

In one embodiment, R⁶, R⁷, R⁸, R⁹, R¹⁰, R¹¹, R¹² are each independently —H, —(C₁-C₁₂)-alkyl, —O—(C₁-C₁₂)-alkyl, —O—(C₆-C₂₀)-aryl, —(C₆-C₂₀)-aryl, —S-alkyl, or —S-aryl.

In one embodiment, R¹³, R¹⁴, R¹⁵, R¹⁶, R¹⁷, R¹⁸, R¹⁹ are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

In one embodiment, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

In one embodiment, R²⁹, R³⁰, R³¹, R³², R³³ are each independently —H, —(C₁-C₁₂)-alkyl, —O—(C₁-C₁₂)-alkyl or —O—(C₆-C₂₀)-aryl.

In one embodiment, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

In one embodiment, R¹, R², R³, R⁴, R⁵ are each independently —H, —(C₁-C₁₂)-alkyl, —O—(C₁-C₁₂)-alkyl or —O—(C₆-C₂₀)-aryl.

In one embodiment, R⁶, R⁷, R⁸, R⁹, R¹⁰, R¹¹, R¹² are each independently —H, —(C₁-C₁₂)-alkyl, —O—(C₁-C₁₂)-alkyl or —O—(C₆-C₂₀)-aryl.

In one embodiment, R¹³, R¹⁴, R¹⁵, R¹⁶, R¹⁷, R¹⁸, R¹⁹ are each independently —H, —(C₁-C₁₂)-alkyl, —O—(C₁-C₁₂)-alkyl or —O—(C₆-C₂₀)-aryl.

In one embodiment, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl or —O—(C₆-C₂₀)-aryl.

In one embodiment, R²⁹, R³⁰, R³¹, R³², R³³ are each independently —H, —(C₁-C₁₂)-alkyl or —O—(C₁-C₁₂)-alkyl.

In one embodiment, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl or —O—(C₆-C₂₀)-aryl.

In one embodiment, R¹, R², R³, R⁴, R⁵ are each independently —H, —(C₁-C₁₂)-alkyl or —O—(C₁-C₁₂)-alkyl.

In one embodiment, R⁶, R⁷, R⁸, R⁹, R¹⁰, R¹¹, R¹² are each independently —H, —(C₁-C₁₂)-alkyl or —O—(C₁-C₁₂)-alkyl.

In one embodiment, R¹³, R¹⁴, R¹⁵, R¹⁶, R¹⁷, R¹⁸, R¹⁹ are each independently —H, —(C₁-C₁₂)-alkyl or —O—(C₁-C₁₂)-alkyl.

In one embodiment, R²⁰, R²¹ R²², R²³, R²⁴, R²⁵, R²⁶, R²⁷, R²⁸ are each independently —H, —(C₁-C₁₂)-alkyl or —O—(C₁-C₁₂)-alkyl.

In one embodiment, R²⁹, R³⁰, R³¹, R³², R³³ are each independently —H or —(C₁-C₁₂)-alkyl.

In one embodiment, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —(C₁-C₁₂)-alkyl or —O—(C₁-C₁₂)-alkyl.

In one embodiment, the compound may be of structure (I).

In one embodiment, the compound may be of structure (II).

In one embodiment, the compound may be of structure (III).

In one embodiment, the compound may be of structure (IV).

In one embodiment, the compound may be of structure (V).

In one embodiment, the compound may be one of structures (1) to (7).

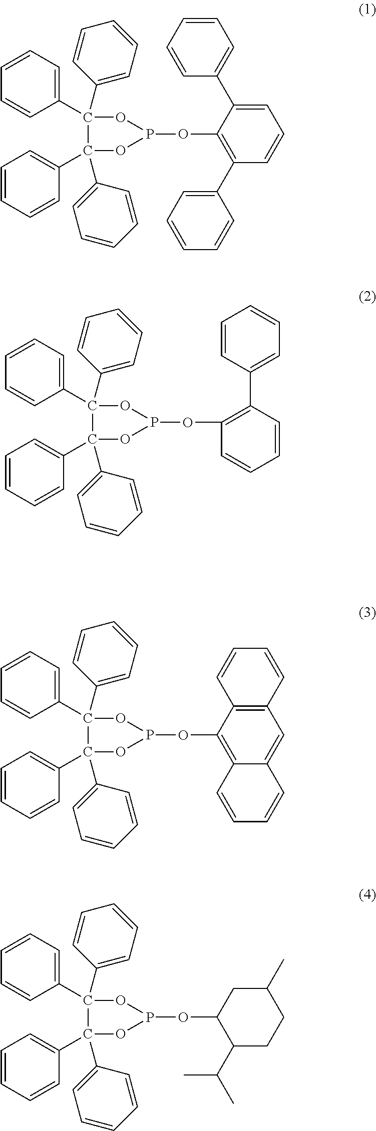 [SMILES: c1ccc(-c2cccc(-c3ccccc3)c2OP2OC(c3ccccc3)(c3ccccc3)C(c3ccccc3)(c3ccccc3)O2)cc1 ; c1ccc(-c2ccccc2OP2OC(c3ccccc3)(c3ccccc3)C(c3ccccc3)(c3ccccc3)O2)cc1 ; c1ccc(C2(c3ccccc3)OP(Oc3c4ccccc4cc4ccccc34)OC2(c2ccccc2)c2ccccc2)cc1 ; CC1CCC(C(C)C)C(OP2OC(c3ccccc3)(c3ccccc3)C(c3ccccc3)(c3ccccc3)O2)C1]

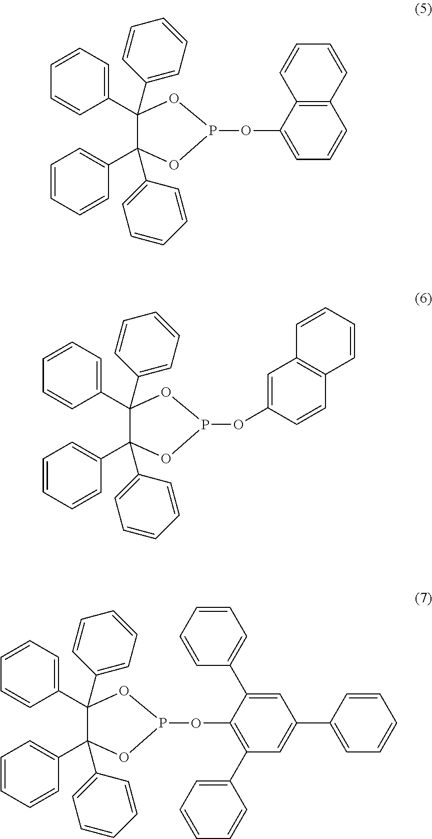 [SMILES: c1ccc(C2(c3ccccc3)OP(Oc3cccc4ccccc34)OC2(c2ccccc2)c2ccccc2)cc1 ; c1ccc(C2(c3ccccc3)OP(Oc3ccc4ccccc4c3)OC2(c2ccccc2)c2ccccc2)cc1 ; c1ccc(-c2cc(-c3ccccc3)c(OP3OC(c4ccccc4)(c4ccccc4)C(c4ccccc4)(c4ccccc4)O3)c(-c3ccccc3)c2)cc1]

In further embodiments the present invention includes a metal complex of a compound as described above and a metal atom selected from: Rh, Ru, Co, Ir.

In one preferred embodiment, the metal may be Rh.

In this regard, see R. Franke, D. Selent, A. Börner, “Applied Hydroformylation”, Chem. Rev., 2012, DOI:10.1021/cr3001803; p. 5688, Scheme 12 “General Method for the Preparation of a P-Modified Rh precatalyst” and references cited therein, and also P. W. N. M. van Leeuwen, in Rhodium Catalyzed Hydroformylation, P. W. N. M. van Leeuwen, C. Claver (eds.), Kluwer, Dordrecht, 2000, inter alia p. 48 ff., p. 233 ff. and references cited therein, and also K. D. Wiese and D. Obst in Top. Organomet. Chem. 2006, 18, 1-13; Springer Verlag Berlin Heidelberg 2006 p. 6 ff. and references cited therein.

The present invention also includes the use of a compound as described above as a ligand in a ligand-metal complex for catalysis of a hydroformylation reaction.

A process in which the compound is used as ligand in a ligand-metal complex for conversion of an olefin to an aldehyde is also an embodiment of the present invention.

Thus in one embodiment the present invention includes a process comprising the following process operations:

a) initially charging an olefin;

b) adding a metal monophosphite complex according to any of the above embodiments, or a monophosphite compound of the structure of the above embodiments and a substance including a metal atom selected from: Rh, Ru, Co, Ir.

c) feeding in H₂ and CO to form a reaction mixture; and

d) heating the reaction mixture to effect conversion of the olefin to an aldehyde.

In this process, process operations a) to d) may be conducted in any desired sequence.

In a preferred variant of the process, the metal atom may be Rh.

In another variant of the process an excess of ligands may be used and each ligand may not necessarily be bound in the form of a ligand-metal complex but may be present as free ligand in the reaction mixture.

The reaction may be conducted under conventionally known conditions.

In one preferred set of conditions the reaction temperature may be from 80° C. to 200° C. and the pressure may be from 1 bar to 300 bar.

In a more preferred set of conditions the temperature may be from 100° C. to 160° C. and the pressure from 15 bar to 250 bar.

The reactants for the hydroformylation in the process of the invention may be olefins or mixtures of olefins, especially monoolefins having 2 to 24, preferably 3 to 16 and more preferably 3 to 12 carbon atoms, having terminal or internal C—C double bonds, for example 1-propene, 1- or 2-pentene, 2-methyl-1-butene, 2-methyl-2-butene, 3-methyl-1-butene, 1-, 2- or 3-hexene, the C₆ olefin mixture obtained in the dimerization of propene (dipropene), heptenes, 2- or 3-methyl-1-hexenes, octenes, 2-methylheptenes, 3-methylheptenes, 5-methyl-2-heptene, 6-methyl-2-heptene, 2-ethyl-1-hexene, the C₈ olefin mixture obtained in the dimerization of butenes (dibutene), nonenes, 2- or 3-methyloctenes, the C₉ olefin mixture obtained in the trimerization of propene (tripropene), decenes, 2-ethyl-1-octene, dodecenes, the C₁₂ olefin mixture obtained in the tetramerization or the trimerization of butenes (tetrapropene or tributene), tetradecenes, hexadecenes, the C₁₆ olefin mixture obtained in the tetramerization of butenes (tetrabutane), and olefin mixtures prepared by cooligomerization of olefins having different numbers of carbon atoms (preferably 2 to 4).

Having generally described this invention, a further understanding can be obtained by reference to certain specific examples which are provided herein for purposes of illustration only, and are not intended to be limiting unless otherwise specified.

### Examples

The invention is illustrated in detail hereinafter by working examples.

### General Procedures

All the preparations which follow were carried out under protective gas using standard Schlenk techniques. The solvents were dried over suitable desiccants before use (Purification of Laboratory Chemicals, W. L. F. Armarego (Author), Christina Chai (Author), Butterworth Heinemann (Elsevier), 6th edition, Oxford 2009).

Phosphorus trichloride (Aldrich) was distilled under argon before use. All preparative operations were effected in baked-out vessels. The products were characterized by NMR spectroscopy. Chemical shifts (δ) are reported in ppm. The ³¹P NMR signals were referenced as follows: SR_{31P}=SR_{1H}*(BF₃₁ₚ/BF_{1H})=SR_{1H}*0.4048. (Robin K. Harris, Edwin D. Becker, Sonia M. Cabral de Menezes, Robin Goodfellow, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Biphenols

The biphenols were synthesized analogously to methods described in DE102013203865 and DE102013203867.

### Synthesis of 2-chloro-4,4,5,5-tetraphenyl-1,3,2-dioxaphospholane

2-Chloro-4,4,5,5-tetraphenyl-1,3,2-dioxaphospholane was synthesized as described in WO 2009/146984 or “A New Diphosphite Promoting Highly Regioselective Rhodium-Catalyzed Hydroformylation” by Detlef Selent, Robert Franke, Christoph Kubis, Anke Spannenberg, Wolfgang Baumann, Burkard Kreidler and Armin Börner in Organometallics 2011, 30, 4509-4514.

To a suspension of 1.878 g (5.12 mmol) of benzopinacol (Acros, 98%) in 30 ml of tetrahydrofuran (Sigma-Aldrich, 99.9%) were added dropwise, at −40° C. while stirring, a 0.768 molar solution of phosphorus trichloride (Aldrich, 99%) in tetrahydrofuran (10.7 ml; 8.22 mmol) and then a solution of 1.56 g (15.4 mmol) of triethylamine (Aldrich, p.a.) in 4 ml of tetrahydrofuran, forming a voluminous colourless precipitate. The mixture was allowed to warm to room temperature and was stirred for a further 5 h. The reaction mixture was filtered through a G4 frit and the filtrate was dried at 40° C. and 20 mbar for 1.5 h. The viscous residue was taken up in toluene (15 ml). The solution was filtered through a G4 frit, and the filtrate was concentrated at 20 mbar and then dried at bath temperature 40° C. and 10⁴ mbar for 2 h. 2.32 g (90% of theory, calculated as toluene adduct) of a high-viscosity liquid which still contained 0.8 equivalent of toluene were obtained.

Analysis: ³¹P{1H}-NMR (C₆D₆) δ=173.44 ppm.

### Synthesis of the Monophosphites

### 2-([1,1′:3′,1″-Terphenyl]-2′-yloxy)-4,4,5,5-tetraphenyl-1,3,2-dioxaphospholane

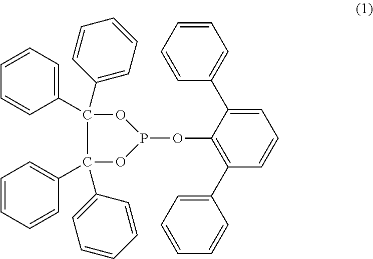 [SMILES: c1ccc(-c2cccc(-c3ccccc3)c2OP2OC(c3ccccc3)(c3ccccc3)C(c3ccccc3)(c3ccccc3)O2)cc1]

To a solution of 2,6-diphenylphenol (0.614 g; 2.493 mmol) in THF (5 ml) was added one equivalent of n-butyllithium dissolved in hexane (8 ml) at −20° C. The mixture was stirred at −20° C. for 20 min and warmed to room temperature. Then a solution of 2-chloro-4,4,5,5-tetraphenyl-1,3,2-dioxaphospholane (1.074 g; 2.493 mmol) in THF (6 ml) was added dropwise. The reaction mixture was stirred at room temperature overnight. The solvent was drawn off under reduced pressure, then toluene (15 ml) was added and the resulting solution was filtered twice. The filtrate was concentrated to dryness. The pale yellow solid obtained was dried at 50° C. for 4 h. Yield: 1.448 g (2.260 mmol; 91%).

Elemental analysis (calculated for C₄₄H₃₃O₃P=640.72 g/mol) C, 82.32 (82.48); H, 5.39 (5.19); P, 4.94 (4.84)%.

³¹P NMR (CD₂Cl₂): 144.7 ppm.

¹H NMR (CD₂Cl₂): 87 (m, 4H); 6.99 (m, 4H); 7.08 (m, 2H); 7.22-7.30 (m, 6H); 7.30-7.37 (m, 9H); 7.37-7.45 (m, 8H) ppm.

¹³C NMR (CD₂Cl₂): 94.9 (d, J_{CP}=8.1 Hz); 125.0; 127.2; 127.3; 127.4; 127.5; 127.7; 128.6; 128.9; 129.0; 130.5; 130.5; 130.8; 136.6 (d, J_{CP}=4.7 Hz); 138.5; 142.2 (d, J_{CP}=4.6 Hz); 142.3; 147.0 (d, J_{CP}=9.1 Hz) ppm.

ESI-TOF/HRMS: m/e 641.22372 (M+H)⁺.

### 2-([1,1′-Biphenyl]-2-yloxy)-4,4,5,5-tetraphenyl-1,3,2-dioxaphospholane

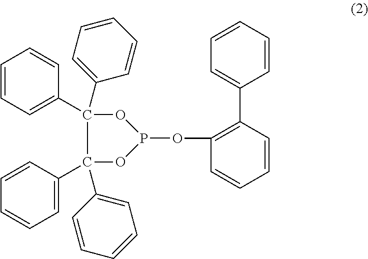 [SMILES: c1ccc(-c2ccccc2OP2OC(c3ccccc3)(c3ccccc3)C(c3ccccc3)(c3ccccc3)O2)cc1]

To a solution of o-phenylphenol (0.253 g; 1.487 mmol) in toluene (3 ml) was added triethylamine (0.452 g; 4.463 mmol). The mixture was cooled to 0° C. and added dropwise to a solution, cooled to 0° C., of 2-chloro-4,4,5,5-tetraphenyl-1,3,2-dioxaphospholane (0.641 g; 1.488 mmol) in toluene (5 ml) The reaction mixture was stirred overnight and then filtered. The filtrate was concentrated to dryness under reduced pressure. Yield: 0.808 g (1.430 mmol; 96%).

Elemental analysis (calculated for C₃₈H₂₉O₃P=564.58 g/mol) C, 80.47 (80.83); H, 5.07 (5.18); P, 5.59 (5.49)%.

³¹P NMR (CD₂Cl₂): 139.0 ppm.

¹H NMR (CD₂Cl₂): 7.01-7.27 (m, 17H); 7.27-7.32 (m, 2H); 7.33-7.45 (m, 6H); 7.54 (m, 4H) ppm.

¹³C NMR (CD₂Cl₂): 95.7 (d, J_{CP}=8.4 Hz); 121.7 (d, J_{CP}=12.9 Hz); 124.8; 127.5 (d, J_{CP}=13.9 Hz); 127.6 (d, J_{CP}=18.5 Hz); 128.4; 129.1 (d, J_{CP}=4.3 Hz); 130.1; 130.3; 131.2; 134.2 (d, J_{CP}=2.9 Hz); 140.0; 142.3 (d, J_{CP}=4.7 Hz); 142.8; 148.8 (d, J_{CP}=9.2 Hz) ppm.

ESI-TOF/HRMS: m/e 565.19253 (M+H)⁺.

### 2-(Anthracen-9-yloxy)-4,4,5,5-tetraphenyl-1,3,2-dioxaphospholane

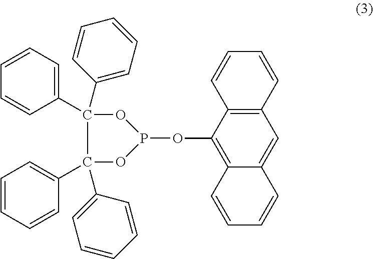 [SMILES: c1ccc(C2(c3ccccc3)OP(Oc3c4ccccc4cc4ccccc34)OC2(c2ccccc2)c2ccccc2)cc1]

To a stirred suspension of anthracen-9-ol (0.252 g; 1.296 mmol) in THF (3 ml) was added one equivalent of n-butyllithium dissolved in hexane (4 ml) at −20° C. The orange mixture was stirred at −20° C. for 20 min and warmed to room temperature. Then a solution of 2-chloro-4,4,5,5-tetraphenyl-1,3,2-dioxaphospholane (0.559 g; 1.296 mmol) in THF (3 ml) was added dropwise. The yellow reaction mixture was stirred at room temperature overnight and then the solvent was removed under reduced pressure. Toluene (10 ml) was added and the resulting solution was filtered. The filtrate was concentrated to dryness. The resulting solid was dried at 50° C./0.1 mbar and purified by column chromatography (hexane/dichloromethane, 2:1, R_{f}=0.5). Yield: 0.554 g (0.942 mmol; 73%).

Elemental analysis (calculated for C₄₀H₂₉O₃P=588.64 g/mol) C, 81.56 (81.62); H, 5.13 (4.97) %.

³¹P NMR (CD₂Cl₂): 144.6 ppm.

¹H NMR (CD₂Cl₂): 7.11-7.30 (m, 12H); 7.30-7.41 (m, 7H); 7.53 (m, 3H); 7.91 (m, 3H); 8.05 (m, 3H); 8.31 (m, 1H) ppm.

¹³C NMR (CD₂Cl₂): 96.2 (d, J_{CP}=8.1 Hz); 122.8; 123.2; 125.0 (d, J_{CP}=3.9 Hz); 126.0 (d, J_{CP}=6.0 Hz); 127.5; 127.6; 127.9; 128.1; 128.4; 128.5; 129.2 (d, J_{CP}=3.4 Hz); 130.6; 132.5; 139.3; 142.2 (d, J_{CP}=4.3 Hz); 143.0; 143.6 (d, J_{CP}=7.2 Hz) ppm.

ESI-TOF/HRMS: m/e 589.19277 (M+H)⁺.

### 2-((2-Isopropyl-5-methylcyclohexyl)oxy)-4,4,5,5-tetraphenyl-1,3,2-dioxaphospholane

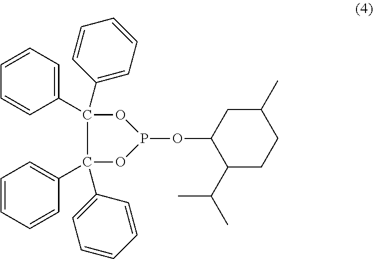 [SMILES: CC1CCC(C(C)C)C(OP2OC(c3ccccc3)(c3ccccc3)C(c3ccccc3)(c3ccccc3)O2)C1]

To a solution of (−)-menthol (0.225 g; 1.442 mmol) in THF (3 ml) was added one equivalent of n-butyllithium dissolved in hexane (4.5 ml) at −20° C. The mixture was stirred at this temperature for 20 min and warmed to room temperature. Then a solution of 2-chloro-4,4,5,5-tetraphenyl-1,3,2-dioxaphospholane (0.621 g; 1.442 mmol) in THF (4 ml) was added dropwise, in the course of which the colour changed from blue to green to yellow. The reaction mixture was stirred overnight and the solvent was removed under reduced pressure. Toluene (10 ml) was added and the resulting solution was filtered. The filtrate was concentrated to dryness and the resulting residue was dried under reduced pressure at room temperature. Yield: 0.710 g (1.289 mmol; 89%).

Elemental analysis (calculated for C₃₆H₃₉O₃P=550.68 g/mol) C, 78.50 (78.52); H, 7.23 (7.14); P, 5.61 (5.62)%.

³¹P NMR (CD₂Cl₂): 147.8 ppm.

¹H NMR (CD₂Cl₂): 0.78 (m, 7H); 0.89 (m, 3H); 0.92-1.03 (m, 2H); 1.27-1.51 (m, 2H); 1.55-1.69 (m, 2H); 1.69-1.78 (m, 1H); 1.78-1.83 (m, 1H); 3.83-3.98 (m, 1H); 7.00-7.28 (m, 16H. Hₐᵣₒₘ); 7.47-7.56 (m, 4H, Hₐᵣₒₘ) ppm.

¹³C NMR (CD₂Cl₂): 15.7; 21.1; 22.2; 23.1; 25.3; 26.0; 32.0; 34.5; 44.4; 48.7; 68.2; 75.0 (D, J_{CP}=21.1 HZ); 94.5 (D, J_{CP}=8.3 HZ); 94.7 (D, J_{CP}=8.0 HZ); 127.2; 127.3; 127.4; 127.4; 129.1 (d, J_{CP}=3.3 Hz); 129.2 (d, J_{CP}=3.3 Hz); 130.4 (d, J_{CP}=5.4 Hz); 142.9; 143.0; 143.2; 143.6 ppm.

### 2-(Naphthyl-1-oxy)-4,4,5,5-tetraphenyl-1,3,2-dioxaphospholane

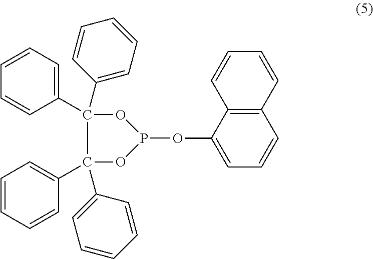 [SMILES: c1ccc(C2(c3ccccc3)OP(Oc3cccc4ccccc34)OC2(c2ccccc2)c2ccccc2)cc1]

To a solution of 1-naphthol (0.231 g; 1.600 mmol) in THF (4 ml) was added one equivalent of n-butyllithium dissolved in hexane (5 ml) at −20° C. The cloudy mixture was stirred at −20° C. for 20 min and warmed to room temperature. Then a solution of 2-chloro-4,4,5,5-tetraphenyl-1,3,2-dioxaphospholane (0.689 g; 1.600 mmol) in THF (3 ml) was added dropwise. The reaction mixture was stirred at room temperature overnight and then the solvent was removed under reduced pressure. Toluene (10 ml) was added and the resulting solution was filtered. The filtrate was concentrated to dryness. The resulting solid was dried at room temperature/0.1 mbar for 5 h. Yield: 0.660 g (1.227 mmol; 77%).

Elemental analysis (calculated for C₃₆H₂₇O₃P=538.16 g/mol) C, 80.62 (80.35); H, 4.97 (4.98); P, 5.74 (5.75)%.

³¹P NMR (CD₂Cl₂): 139.3 ppm.

¹H NMR (CD₂Cl₂): 7.09-7.26 (m, 11H); 7.26-7.36 (m, 6H); 7.37-7.60 (m, 4H); 7.61-7.69 (m, 1H); 7.69-7.81 (m, 4H); 7.81-7.92 (m, 1H) ppm.

¹³C NMR (CD₂Cl₂): 95.8 (d, J_{CP}=8 Hz), 115.1 (d, J_{CP}=14 Hz), 122.8; 124.2; 126.0 (d, J_{CP}=9 Hz); 126.9; 127.5; 127.7; 127.9; 128.6; 129.1 (d, J_{CP}=3 Hz); 129.4; 130.4; 135.1; 142.3 (d, J_{CP}=4 Hz); 143.0; 147.9 (d, J_{CP}=9 Hz) ppm.

ESI-TOF/HRMS: m/e 577.13377 (M+K)⁺.

### 2-(Naphthyl-2-oxy)-4,4,5,5-tetraphenyl-1,3,2-dioxaphospholane

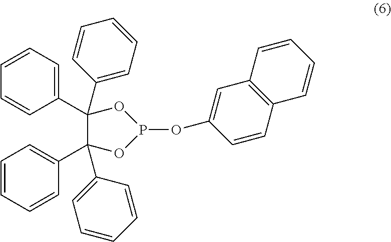 [SMILES: c1ccc(C2(c3ccccc3)OP(Oc3ccc4ccccc4c3)OC2(c2ccccc2)c2ccccc2)cc1]

To a solution of 2-naphthol (0.231 g; 1.600 mmol) in THF (4 ml) was added one equivalent of n-butyllithium dissolved in hexane (5 ml) at −20° C. The yellow mixture was stirred at −20° C. for 20 min and warmed to room temperature. Then a solution of 2-chloro-4,4,5,5-tetraphenyl-1,3,2-dioxaphospholane (0.689 g; 1.600 mmol) in THF (3 ml) was added dropwise; after 20 min, the mixture turned cloudy. The reaction mixture was stirred at room temperature overnight and then the solvent was removed under reduced pressure. Toluene (10 ml) was added and the resulting solution was filtered. The filtrate was concentrated to dryness. The resulting solid was dried at room temperature/0.1 mbar for 5 h. Yield: 0.861 g (1.600 mmol; 100%). Elemental analysis (calculated for C₃₆H₂₇O₃P=538.16 g/mol) C, 80.57 (80.35); H, 4.95 (4.98); P, 5.77 (5.75)%.

³¹P NMR (CD₂Cl₂): 138.5 ppm.

¹H NMR (CD₂Cl₂): 6.88-7.00 (m, 1H); 7.07-7.25 (m, 10H); 7.25-7.39 (m, 7H); 7.41-7.60 (m, 2H); 7.61-7.74 (m, 4H); 7.74-7.92 (m, 3H) ppm.

¹³C NMR (CD₂Cl₂): 95.6 (d, J_{CP}=8 Hz), 116.6 (d, J_{CP}=11 Hz), 121.6 (d, J_{CP}=6 Hz); 125.2; 125.7; 126.9; 127.4; 127.6; 127.7; 127.8. 128.0; 128.6. 129.1 (d, J_{CP}=3 Hz); 129.4; 130.0; 130.3; 130.8; 134.4; 142.3 (d, J_{CP}=4 Hz); 143.0; 149.2 (d, J_{CP}=8 Hz) ppm.

ESI-TOF/HRMS: m/e 577.13431 (M+K)⁺.

### 4,4,5,5-Tetraphenyl-2-((5′-phenyl-[1,1′:3′,1″-terphenyl]-4′-yl)oxy)-1,3,2-dioxaphospholane

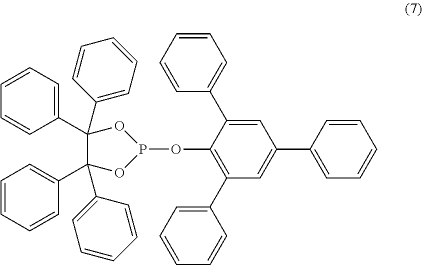 [SMILES: c1ccc(-c2cc(-c3ccccc3)c(OP3OC(c4ccccc4)(c4ccccc4)C(c4ccccc4)(c4ccccc4)O3)c(-c3ccccc3)c2)cc1]

To a solution, cooled to −20° C., of 2,4,6-triphenylphenol (0.5 g; 1.55 mmol) in THF (12 ml) is added dropwise n-BuLi (1 ml of a 1.6 M solution in hexane=1.6 mmol). The mixture is allowed to warm to room temperature and then a suspension of 2-chloro-4,4,5,5-tetraphenyl-1,3,2-dioxaphospholane (0.667 g; 1.55 mmol) in THF (6 ml) is added. The mixture is stirred at room temperature for 24 h, the solvent is removed under reduced pressure and toluene (8 ml) is added. The mixture is filtered. The filtrate is concentrated to dryness and the residue is purified by column chromatography with toluene as eluent. Yield: 0.201 g (0.279 mmol; 18%).

³¹P NMR (CD₂Cl₂): δ 144.1 ppm.

¹H NMR (CD₂Cl₂): δ 7.67-6.81 (m, 37H) ppm.

¹³C NMR (75.5 MHz, CD₂Cl₂): δ=149.5; 142.6; 142.2 (d, J_{CP}=4.5); 140.9; 140.6; 138.5; 138.0; 137.8; 136.8; 136.8; 134.1; 130.8; 130.5; 129.8; 129.6; 129.4; 129.3; 129.2; 129.1; 128.9; 129.0; 128.6; 128.4; 128.1; 128.0; 127.7; 127.7; 127.4; 127.3; 127.2; 127.1; 125.6; 94.8 (d, J_{CP}=8.7 Hz) ppm.

### 4,4,5,5-Tetraphenyl-2-(2,4,6-tri-tert-butylphenoxy)-1,3,2-dioxaphospholane

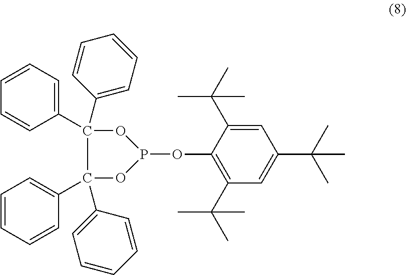 [SMILES: CC(C)(C)c1cc(C(C)(C)C)c(OP2OC(c3ccccc3)(c3ccccc3)C(c3ccccc3)(c3ccccc3)O2)c(C(C)(C)C)c1]

To a solution of 2,4,6-tri-tert-butylphenol (0.315 g; 1.201 mmol) in THF (3 ml) was added one equivalent of n-butyllithium dissolved in hexane (4 ml) at room temperature. After stirring for 10 minutes, the pink/yellow suspension obtained was added dropwise to a solution of 2-chloro-4,4,5,5-tetraphenyl-1,3,2-dioxaphospholane (0.518 g; 1.203 mmol) in THF (3 ml). The reaction mixture was stirred at −20° C. for 10 min and at room temperature overnight. The solvent was removed under reduced pressure, toluene (15 ml) was added and the resulting solution was filtered. The filtrate was concentrated to dryness. The resulting solid was dried at 40° C./0.1 mbar for 1 h. Recrystallization was effected first from hot hexane (26 ml) and then from acetonitrile (2 ml). Yield: 0.205 g (0.312 mmol; 26%).

³¹P NMR (CD₂Cl₂): 145.8 ppm.

¹H NMR (CD₂Cl₂): 1.42 (s, 9H); 1.47 (s, 18H); 7.06-7.33 (m, 16H, Hₐᵣₒₘ); 7.45 (s, 2H, Hₐᵣₒₘ); 7.71-7.76 (m, 4H, Hₐᵣₒₘ) ppm.

¹³C NMR (CD₂Cl₂): 31.7; 32.0; 32.0; 35.0; 35.7; 96.1 (d, J_{CP}=9.3 Hz); 123.6; 127.4; 127.4; 127.7; 127.8; 129.1 (d, J_{CP}=4.3 Hz); 130.7; 142.4 (d, J_{CP}=4.8 Hz); 142.6; 143.1 (d, J_{CP}⁼3.2 Hz); 145.9; 148.2 (d, J_{CP}=11.8 Hz) ppm.

ESI-TOF/HRMS: m/e 657.34885 (M+H)⁺.

### 8-((4,4,5,5-Tetraphenyl-1,3,2-dioxaphospholan-2-yl)oxy)quinoline

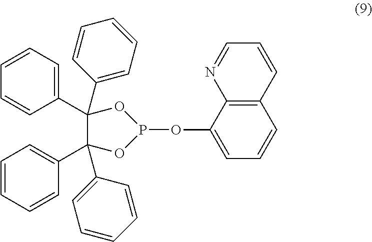 [SMILES: c1ccc(C2(c3ccccc3)OP(Oc3cccc4cccnc34)OC2(c2ccccc2)c2ccccc2)cc1]

To a solution of quinolin-8-ol (0.232 g; 1.600 mmol) in THF (4 ml) was added one equivalent of n-butyllithium dissolved in hexane (5 ml) at −20° C. The yellow mixture was stirred at −20° C. for 20 min and warmed to room temperature. Then a solution of 2-chloro-4,4,5,5-tetraphenyl-1,3,2-dioxaphospholane (0.689 g; 1.600 mmol) in THF (3 ml) was added dropwise; after 15 min, precipitate formed. The reaction mixture was stirred at room temperature overnight and then the solvent was removed under reduced pressure. Toluene (10 ml) was added and the suspension was heated, then filtered while hot. The filtrate was concentrated to dryness. The resulting solid was dried at 50° C./0.1 mbar. Then the substance was stirred in hexane (15 ml) and the suspension was filtered. The resulting solid was dried at 50° C./0.1 mbar for 4 h. Yield: 0.760 g (1.409 mmol; 88%).

Elemental analysis (calculated for C₃₅H₂₆O₃PN=539.57 g/mol) C, 77.90 (77.91); H, 4.96 (4.86); P, 5.63 (5.74); N, 2.47 (2.60) %.

³¹P NMR (CD₂Cl₂): 134.8 ppm.

¹H NMR (CD₂Cl₂) 74-6.77 (m, 1H); 7.09-7.14 (m, 2H); 7.14-7.26 (m, 14H); 7.40 (t, 1H); 7.49-7.56 (m, 2H); 7.58-7.64 (m, 4H); 8.21-8.25 (m, 1H); 9.06-9.09 (m, 1H) ppm.

¹³C NMR (CD₂Cl₂): 95.3 (d, J_{CP}=8 Hz), 118.4; 122.1; 122.2; 127.4; 127.5; 127.5; 129.3 (d, J_{CP}=4 Hz); 130.2; 136.3; 142.9 (d, J_{CP}=4 Hz); 143.6; 149.0; 149.8 ppm.

ESI-TOF/HRMS: m/e 540.17262 (M+H)⁺.

### 2-Phenoxy-4,4,5,5-tetraphenyl-1,3,2-dioxaphospholane

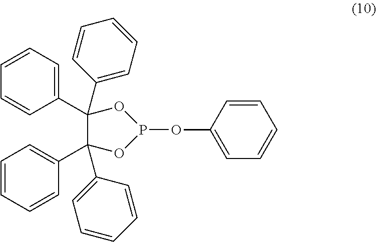 [SMILES: c1ccc(OP2OC(c3ccccc3)(c3ccccc3)C(c3ccccc3)(c3ccccc3)O2)cc1]

To a stirred solution of phenol (0.186 g; 1.98 mmol) in toluene (5 ml) was added, at room temperature, triethylamine (0.301 g; 2.97 mmol) and then, dropwise, a solution of 2-chloro-4,4,5,5-tetraphenyl-1,3,2-dioxaphospholane (0.853 g; 1.98 mmol) in toluene (5 ml). The mixture was stirred overnight and filtered, and the filtrate was concentrated. Yield of crude product: 0.890 g. The crude product was taken up once again in toluene, and the resulting solution was filtered through a layer of silica gel. The filtrate was concentrated to dryness and dried at 50° C./0.1 mbar.

Elemental analysis (calculated for C₃₂H₂₅O₃P=488.15 g/mol): C, 78.48 (78.68); 5.19 (5.16); P, 6.30 (6.34) %.

³¹P NMR (121 MHz, CD₂Cl₂): δ 138.5 ppm.

¹H NMR (CD₂Cl₂): δ 7.58-7.50 (m, 4H), 7.27-7.01 (m. 19H), 6.75-6.69 (m, 2H), 1.46 ppm.

¹³C NMR (CD₂Cl₂): δ 151.5 (d, J_{CP}=8.7 Hz). 143.0; 142.3 (d_{CP}, J=4.2 Hz). 130.2; 129.9; 129.1 (d, J_{CP}=3.3 Hz), 127.7 (d, J_{CP}=16.7 Hz), 127.6; 127.4, 124.3, 121.0 (d, J_{CP}=8.2 Hz), 95.5 (d, J_{CP}=8.2 Hz) ppm.

### 2-([1,1′-Biphenyl]-3-yloxy)-4,4,5,5-tetraphenyl-1,3,2-dioxaphospholane

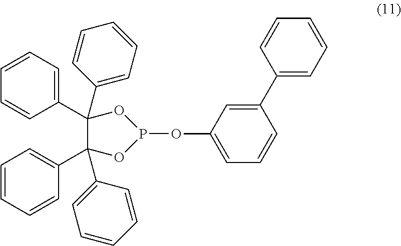 [SMILES: c1ccc(-c2cccc(OP3OC(c4ccccc4)(c4ccccc4)C(c4ccccc4)(c4ccccc4)O3)c2)cc1]

To a stirred solution of 3-phenylphenol (0.373 g; 1.97 mmol) in toluene (6 ml) was added, at room temperature, triethylamine (0.301 g; 2.97 mmol) and then, dropwise, a solution of 2-chloro-4,4,5,5-tetraphenyl-1,3,2-dioxaphospholane (0.850 g; 1.97 mmol) in toluene (6 ml). The mixture was stirred overnight and filtered, and the filtrate was concentrated to dryness under reduced pressure. Yield: 1.07 g of crude product. The crude product was taken up in toluene, and the resulting solution was filtered through a layer of silica gel. The filtrate was concentrated to dryness and dried at 50° C./0.1 mbar.

Elemental analysis (calculated for C₃₈H₂₉O₃P=564.61 g/mol): C, 80.81 (80.84); 5.17 (5.18); P, 5.46 (5.49) %.

³¹P NMR (CD₂Cl₂): δ 138.5 ppm.

¹H NMR (CD₂Cl₂): δ 7.60-7.03 (m, 27H), 6.87-6.74 (m, 2H) ppm.

¹³C NMR (CD₂Cl₂): δ 151.8 (d, J_{CP}=8.1 Hz), 143.1 (d, J_{CP}=5.2 Hz); 142.3 (d, J_{CP}=4.1 Hz); 141.0 (d, J_{CP}=5.0 Hz); 140.6; 130.4; 130.3; 130.2; 129.4; 129.3; 129.2, 129.1, 129.0, 128.7; 128.6; 128.5, 128.5; 128.4; 128.3; 128.0; 127.9; 127.8; 127.7; 127.6; 127.4; 127.2; 125.7; 123.0; 119.8; 119.7; 114.6; 114.3; 97.8; 95.6 (d, J_{CP}=8.0 Hz) ppm.

### 2-([1,1′-Biphenyl]-4-yloxy)-4,4,5,5-tetraphenyl-1,3,2-dioxaphospholane

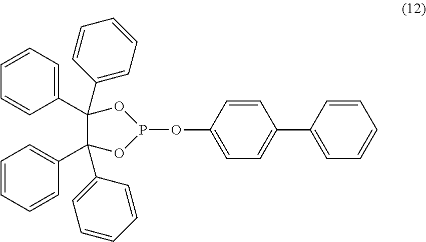 [SMILES: c1ccc(-c2ccc(OP3OC(c4ccccc4)(c4ccccc4)C(c4ccccc4)(c4ccccc4)O3)cc2)cc1]

To a stirred suspension of 4-phenylphenol (0.337 g; 1.98 mmol) in toluene (10 ml) were added, at room temperature, triethylamine (0.301 g; 2.97 mmol) and then, dropwise, a solution of 2-chloro-4,4,5,5-tetraphenyl-1,3,2-dioxaphospholane (0.853 g; 1.98 mmol) in toluene (5 ml). The mixture was stirred overnight and filtered, and the filtrate was filtered once again through a thin layer of silica gel. The filtrate was concentrated to dryness under reduced pressure. Yield: 1.15 g. The crude product was taken up again in toluene (15 ml), and the resulting solution was filtered through a layer of silica gel. Concentration of the filtrate and drying at 0.1 mbar gives pure monophosphite.

Elemental analysis (calculated for C₃₈H₂₉O₃P=564.61 g/mol): C, 80.57 (80.84); 5.26 (5.18); P, 5.39 (5.49) %.

³¹P NMR (CD₂Cl₂): δ 138.4 ppm.

¹H NMR (CD₂Cl₂): δ 7.60-6.80 (m, 27H), 6.79 (m, 2H) ppm.

¹³C NMR (CD₂Cl₂): δ 151.1 (d, J_{CP}=8.4 Hz), 143.1; 142.3 (d, J_{CP}=4.0 Hz), 140.8; 137.4; 130.3; 129.4; 129.1 (d, J_{CP}=3.3 Hz), 128.6; 127.7 (d, J_{CP}=21.0 Hz); 127.7; 127.5; 127.4, 127.2; 125.7; 121.2 (D, J_{CP}=8.1 HZ); 95.6 (D, J_{CP}=7.9 HZ) PPM.

### Procedure for the Catalyst Experiments

The hydroformylation was conducted in a 200 ml autoclave equipped with pressure-retaining valve, gas flow meter, sparging stirrer and pressure pipette from Premex Reactor AG, Lengau, Switzerland. To minimize the influence of moisture and oxygen, the toluene used as solvent was dried with sodium ketyl and distilled under argon. The olefin used was an octene mixture: n-octenes (Oxeno GmbH, octene isomer mixture of 1-octene: ˜3%; cis+trans-2-octene; ˜49%; cis+trans-3-octene: ˜29%; cis+trans-octene-4: ˜16%; structurally isomeric octenes: ˜3%), which was heated at reflux over sodium and distilled over argon for several hours.

For the experiments, the following solutions of rhodium in the form of [(acac)Rh(COD)] (acac=acetylacetonate anion; COD=1,5-cyclooctadiene, Umicore) as the catalyst precursor in toluene were introduced into the autoclave under an argon atmosphere: for experiments at 100 ppm by mass of rhodium 10 ml of a 4.31 millimolar solution, for 40 or 60 ppm by mass the same amount of an appropriately diluted solution. The appropriate amount of the phosphite compound (5 ligand equivalents per unit rhodium) dissolved in toluene was then added. By addition of further toluene (the total amount of toluene was determined for the GC analysis, see below), the initial volume of the catalyst solution was adjusted to 41.0 ml. The mass of toluene introduced was determined in each case. Weight of n-octene: 10.70 g (95.35 mmol). The autoclave was heated to the temperatures stated in each case at a total gas pressure (synthesis gas: Linde; H₂ (99.999%):CO (99.997%)=1:1) of a) 42 bar for a final pressure of 50 bar or b) 12 bar for a final pressure of 20 bar with stirring (1500 rpm). After reaching the reaction temperature, the synthesis gas pressure was increased to a) 48.5 bar for a final pressure of 50 bar or b) 19.5 bar for a final pressure of 20 bar and the reactant was introduced under a positive pressure of about 3 bar set in the pressure pipette. The reaction was conducted at a constant pressure of 50 or 20 bar in each case (closed-loop pressure controller from Bronkhorst, the Netherlands) over 4 h. After the reaction time had elapsed, the autoclave was cooled to room temperature, decompressed while stirring and purged with argon. 1 ml of each reaction mixture was removed immediately after the stirrer had been switched off, diluted with 5 ml of pentane and analysed by gas chromatography. HP 5890 Series II plus, PONA, 50 m×0.2 mm×0.5 μm, the quantitative determination of residual olefins and aldehydes was carried out with reference to the solvent toluene as internal standard.

A comparative example used was the ligand described in WO 2009/146984 or “A New Diphosphite Promoting Highly Regioselective Rhodium-Catalyzed Hydroformylation” by Detlef Selent, Robert Franke, Christoph Kubis, Anke Spannenberg, Wolfgang Baumann, Burkard Kreidler and Armin Borner in Organometallics 2011, 30, 4509-4514 (referred to here as ligand A).

Comparative Ligand:

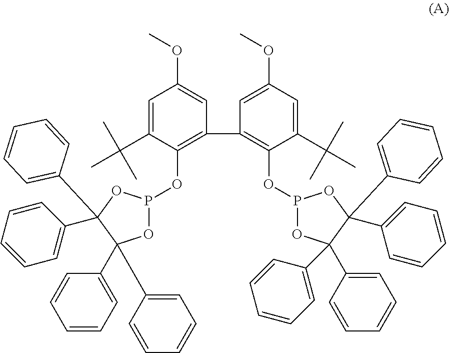 [SMILES: COc1cc(-c2cc(OC)cc(C(C)(C)C)c2OP2OC(c3ccccc3)(c3ccccc3)C(c3ccccc3)(c3ccccc3)O2)c(OP2OC(c3ccccc3)(c3ccccc3)C(c3ccccc3)(c3ccccc3)O2)c(C(C)(C)C)c1]

The ligand was prepared by the methods cited above.

### Catalyst Results

The results of the catalyst experiments are summarized in Tables 1 and 2.

TABLE 1

| | p | T | | [Rh] | | Yield |
|---|---|---|---|---|---|---|
| Ligand | (bar) | (° C.) | t (h) | (ppm) | P/Rh | (%) |
| 1* | 50 | 120 | 4 | 100 | 5 | 97 |
| 2* | 50 | 120 | 4 | 100 | 5 | 99 |
| 3* | 50 | 110 | 4 | 100 | 5 | 97 |
| 3* | 50 | 100 | 4 | 100 | 5 | 90 |
| 3* | 50 | 80 | 4 | 40 | 5 | 92 |
| 4* | 50 | 120 | 4 | 100 | 5 | 93 |
| 5* | 50 | 110 | 4 | 100 | 5 | 98 |
| 6* | 50 | 110 | 4 | 100 | 5 | 94 |
| 7* | 50 | 120 | 4 | 100 | 5 | 89 |
| 8 | 50 | 120 | 4 | 100 | 5 | 18 |
| 9 | 50 | 120 | 4 | 100 | 5 | 2 |
| 10 | 50 | 120 | 4 | 100 | 5 | 40 |
| 11 | 50 | 120 | 4 | 100 | 5 | 9 |
| 12 | 50 | 120 | 4 | 100 | 5 | 3 |
| A | 50 | 120 | 4 | 100 | 5 | 7 |

*inventive compound
Olefin: n-octenes (Oxeno GmbH)
Solvent: toluene
Phosphorus/rhodium ratio (L/Rh): 5:1

The results listed in Table 1 show clearly that the use of the inventive compounds (1) to (7) leads to much better results than the use of the comparative compounds (8) to (12). It was also found that the yields of the inventive monophosphites (1) to (7) are much higher than the yield of the comparative ligand (A) known from the related art.

TABLE 2

| | p | T | | [Rh] | | Yield |
|---|---|---|---|---|---|---|
| Ligand | (bar) | (° C.) | t (h) | (ppm) | P/Rh | (%) |
| 3* | 50 | 80 | 4 | 100 | 5 | 99 |
| A | 50 | 80 | 4 | 100 | 5 | 0.2 |

*inventive compound
Olefin: 2-pentene
Solvent: toluene
Phosphorus/rhodium ratio (L/Rh): 5:1

The compound (3) was tested not just with n-octenes but additionally also with 2-pentene. Here too, a very good result was achievable. With the aid of the comparative ligand (A), it was only possible to generate small traces of target product.

It was shown on the basis of the experiments described above that the stated problem has been solved by the inventive compounds.

### Phosphochloridite of benzopinacole, 2-chloro-4,4′,5,5′-tetraphenyl-1,3,2-dioxaphospholane

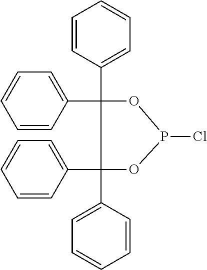 [SMILES: ClP1OC(c2ccccc2)(c2ccccc2)C(c2ccccc2)(c2ccccc2)O1]

A stirred suspension of benzopinacole (3 g, 8.186 mmol) in THF (48 ml) was treated dropwise with solutions of phosphorus trichloride (1.5 mL g, 13.143 mmol) in THF (16 ml) and triethylamine (3.43 mL, 24.63 mmol) in THF (7 ml) at −40° C. The reaction mixture was allowed to warm up slowly to room temperature, then stirred overnight and filtered. Volatiles were removed from the filtrate in vacuo at 40° C. The obtained residue was dissolved in toluene (25 ml). After filtration, solvent was removed in vacuo and the solid product dried at 0.1 bar, 40° C., for 5 h. Yield: 3.5 g (8.12 mmol, 99%). 31P-NMR (CD2Cl2): δ=173.4 (s) ppm. 1H-NMR (CD2C12): 6.99-7.11 (m, 7H), 7.14-7.21 (m, 3H), 7.22-7.32 (m, 6H), 7.51-7.56 (m, 4H). 13C-NMR (CD2Cl2): δ=97.8, (d, 2JCP=8.6 Hz), 126.0, 128.0, 128.4, 128.6, 128.7, 128.8, 128.9, 129.0, 129.3 (d, 4JCP=3.9 Hz), 129.4, 129.6, 129.8, 130.6, 141.6 (d, 3JCP=3.95 Hz), 141.9 ppm.

### 2-phenoxy-4,4′,5,5′-tetraphenyl-1,3,2-dioxaphospholane

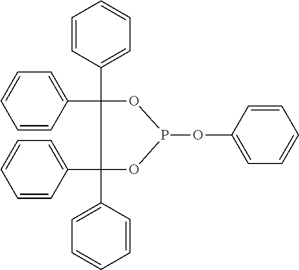 [SMILES: c1ccc(OP2OC(c3ccccc3)(c3ccccc3)C(c3ccccc3)(c3ccccc3)O2)cc1]

To a stirred suspension of Phenol (0.186 g, 1.98 mmol) in Toluene (5 ml), Triethylamine (0.301 g, 2.97 mmol) was added dropwise. The reaction mixture was treated dropwise with solutions of 2-Chloro-4,4,5,5-tetraphenyl-1,3,2-dioxaphospholan (0.853 g; 1.98 mmol) in Toluene (6 ml) at Room temperature. The reaction mixture was stirred overnight and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.610 g (1.2 mmol, 63%). Elemental analysis calc. for C32H25O3P=488.15 g/mol): C, 78.77 (78.68); H, 5.32 (5.16); P, 6.04 (6.34) %. 31P-NMR (CD2Cl2): δ=139.5 (s) ppm. 1H-NMR (CD2Cl2): 6.74-6.79 (m, 2H), 7.05-7.15 (m, 10H), 7.18-7.29 (m, 9H), 7.57-7.61 (m, 4H). 13C-NMR (CD2Cl2): δ=95.9, (d, 2JCP=8.1 Hz), 121.4, (d, JCP=8.1 Hz), 124.7, 127.9, (d, JCP=18.9 Hz), 128.1, (d, JCP=18.9 Hz), 129.5, (d, JCP=3.2 Hz), 130.4, 130.6, 142.7, (d, JCP=4.2 Hz), 143.4, 151.9 (d, JCP=8.81 Hz) ppm. ESI-TOF/HRMS calc. For for C32H25O3P (M+H)+; 489.1614 found; 489.1626.

### 2-([1,1′-Biphenyl]-4-yloxy)-4,4,5,5-tetraphenyl-1,3,2-dioxaphospholan

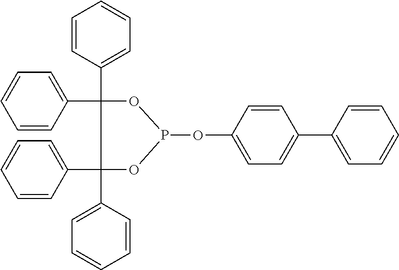 [SMILES: c1ccc(-c2ccc(OP3OC(c4ccccc4)(c4ccccc4)C(c4ccccc4)(c4ccccc4)O3)cc2)cc1]

To a stirred suspension of 4-Phenylphenol (0.373 g, 1.97 mmol) in Toluene (6 ml), Triethylamine (0.301 g, 2.97 mmol) was added dropwise. The reaction mixture was treated dropwise with solutions of 2-Chloro-4,4,5,5-tetraphenyl-1,3,2-dioxaphospholan (0.850 g; 1.97 mmol) in Toluene (6 ml) at Room temperature. The reaction mixture was stirred overnight and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.930 g (1.64 mmol, 83%). Elemental analysis calc. for C38H29O3P=564.61 g/mol): C, 80.95 (80.84); H, 5.20 (5.18); P, 5.31 (5.49) %. 31P-NMR (CD2Cl2): δ=138.9 (s) ppm. 1H-NMR (CD2Cl2): 6.75-6.78 (m, 2H), 7.02-7.11 (m, 10H), 7.15-7.31 (m, 7H), 7.35-7.45 (m, 4H), 7.48-7.55 (m, 6H). 13C-NMR (CD2Cl2): S=95.9, (d, JCP=7.9 Hz), 121.5, (d, JCP=7.9 Hz), 126.0, 127.5, 127.8, 129.0 (d, JCP=21.3 Hz), 128.1, 128.9, 129.4, 129.5, 1306.6, 137.6, 141.1, 142.6 (d, JCP=4.1 Hz), 143.4, (d, JCP=4.1 Hz), 151.3 (d, JCP=8.9 Hz) ppm. ESI-TOF/HRMS calc. For for C32H29O3P (M+H)+; 565.1927 found; 565.1935.

### 2-([1,1′-Biphenyl]-3-yloxy)-4,4,5,5-tetraphenyl-1,3,2-dioxaphospholane

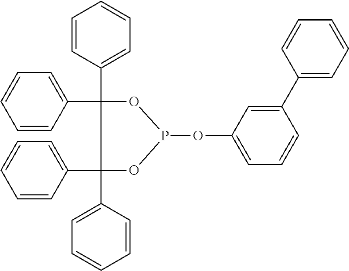 [SMILES: c1ccc(-c2cccc(OP3OC(c4ccccc4)(c4ccccc4)C(c4ccccc4)(c4ccccc4)O3)c2)cc1]

To a stirred suspension of 4-Phenylphenol (0,373 g, 1.97 mmol) in Toluene (6 ml), Triethylamine (0.301 g, 2.97 mmol) was added dropwise. The reaction mixture was treated dropwise with solutions of 2-Chloro-4,4,5,5-tetraphenyl-1,3,2-dioxaphospholan (0,850 g; 1.97 mmol) in Toluene (6 ml) at Room temperature. The reaction mixture was stirred overnight and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.760 g (1.34 mmol, 68%). Elemental analysis calc. for C38H29O3P=564.61 g/mol): C, 80.75 (80.84); H, 5.04 (5.18); P, 5.34 (5.49) %. 31P-NMR (CD2Cl2): δ=139.1 (s) ppm. 1H-NMR (CD2Cl2): 6.76-6.81 (m, 2H), 7.08-7.17 (m, 10H), 7.22-7.27 (m, 6H), 7.31-7.48 (m, 5H), 7.53-7.64 (m, 6H). 13C-NMR (CD2Cl2): δ=95.9, (d, JCP=8.2 Hz), 114.9, 115.3, 119.5, 120.0, 120.1, 120.2, 120.3, 123.4, 126.0, 127.8, 127.9, 128.1, 128.2, 128.4, 128.9, 129.4, 129.5, 129.8, 130.6, 130.7, 140.9, 141.7, 142.6, 142.7, 143.4, 143.5, 152.3 (d, JCP=8.1 Hz) ppm. ESI-TOF/HRMS calc. For for C32H29O3P (M+H)+; 565.1927 found; 565.1929.

### 2-([1,1′-biphenyl]-2-yloxy)-4,4,5,5-tetraphenyl-1,3,2-dioxaphospholane

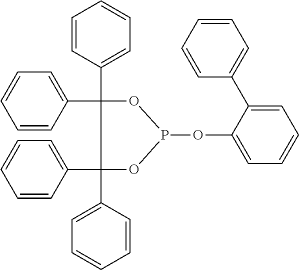 [SMILES: c1ccc(-c2ccccc2OP2OC(c3ccccc3)(c3ccccc3)C(c3ccccc3)(c3ccccc3)O2)cc1]

To a stirred suspension of 2-Phenylphenol (0.374 g, 2.204 mmol) in Toluene (6 ml), Triethylamine (0.301 g, 2.97 mmol) was added dropwise at −20° C. The reaction mixture was treated dropwise with cold solutions of 2-Chloro-4,4,5,5-tetraphenyl-1,3,2-dioxaphospholan (0.950 g; 2.204 mmol) in Toluene (6 ml). The reaction mixture was stirred overnight and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.944 g (1.67 mmol, 76%). Elemental analysis calc. for C38H29O3P=564.61 g/mol): C, 80.76 (80.84); H, 5.20 (5.18); P, 5.23 (5.49) %. 31P-NMR (CD2Cl2): δ=139.3 (s) ppm. 1H-NMR (CD2Cl2): 7.23-7.47 (m, 20H), 7.57-7.62 (m, 5H), 7.77-7.81 (m, 4H). 13C-NMR (CD2Cl2): 8=95.3 (d, JCP=8.3 Hz), 121.3 (d, JCP=12.3 Hz), 124.5, 125.3, 127.2 (d, JCP=13.6 Hz), 127.3 (d, JCP=18.7 Hz), 128.1, 128.4 (d, JCP=19.4 Hz), 128.7 (d, JCP=3.1 Hz), 129.0, 129.8, 129.9, 130.9, 133.9 (d, JCP=3.1 Hz), 137.6, 137.9, 142.0 (d, JCP=5.1 Hz), 142.4, 148.5 (d, JCP=9.1 Hz), 153.2 ppm. ESI-TOF/HRMS calc. For for C38H29O3P (M+H)+; 565.1927 found; 565.1930.

### 2-([1,1′:3′,1″-terphenyl]-2′-yloxy)-4,4,5,5-tetraphenyl-1,3,2-dioxaphospholane

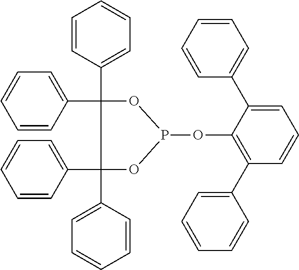 [SMILES: c1ccc(-c2cccc(-c3ccccc3)c2OP2OC(c3ccccc3)(c3ccccc3)C(c3ccccc3)(c3ccccc3)O2)cc1]

To a stirred suspension of 2,6-di-Phenylphenol (0.542 g, 2.204 mmol) in Toluene (6 ml), Triethylamine (0.301 g, 2.97 mmol) was added dropwise at −20° C. The reaction mixture was treated dropwise with cold solutions of 2-Chloro-4,4,5,5-tetraphenyl-1,3,2-dioxaphospholan (0.950 g; 2.204 mmol) in Toluene (6 ml). The reaction mixture was stirred 24 hrs and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 1.113 g (1.74 mmol, 87%). Elemental analysis calc. for C38H29O3P=564.61 g/mol): C, 81.91 (82.48); H, 5.33 (5.19); P, 4.94 (4.84) %. 31P-NMR (CD2Cl2): δ=144.8 (s) ppm. 1H-NMR (CD2Cl2): 6.93-7.17 (m, 9H), 7.28-7.48 (m, 20H), 7.53-7.59 (m, 2H), 7.65-7.69 (m, 2H). 13C-NMR (CD2Cl2): δ=95.0 (d, JCP=8.4 Hz), 121.3, 125.1, 125.9, 127.5 (d, JCP=11.6 Hz), 127.6 (d, JCP=19.3 Hz), 128.0 (d, JCP=22.6 Hz), 128.8, 129.1 (d, JCP=3.4 Hz), 129.4, 129.9, 130.6, 130.9, 136.7 (d, JCP=5.1 Hz), 138.3, 138.7, 142.4 (d, JCP=4.8 Hz), 142.8, 147.2 (d, JCP=9.2 Hz), 150.1 ppm. ESI-TOF/HRMS calc. For for C44H33O3P (M+H)+; 641.2240 found; 641.2244.

### 2-(4-(tert-butyl)phenoxy)-4,4,5,5-tetraphenyl-1,3,2-dioxaphospholane

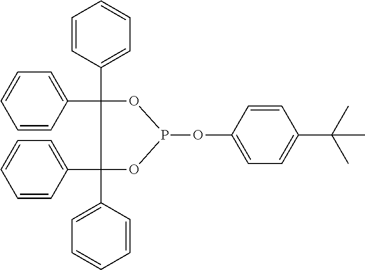 [SMILES: CC(C)(C)c1ccc(OP2OC(c3ccccc3)(c3ccccc3)C(c3ccccc3)(c3ccccc3)O2)cc1]

To a stirred suspension of 4-(tert-butyl)phenol (0.180 g, 1.201 mmol) in Toluene (5 ml), Triethylamine (0.301 g, 2.97 mmol, 4 mL) was added dropwise at room temperature. The reaction mixture was treated dropwise with cold solutions of 2-Chloro-4,4,5,5-tetraphenyl-1,3,2-dioxaphospholan (0.517 g; 1.201 mmol) in Toluene (5 ml). The reaction mixture was stirred 24 hrs at room temperature and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.610 g (1.12 mmol, 93%). Elemental analysis calc. for C36H33O3P=544.63 g/mol): C, 79.50 (79.39); H, 6.18 (6.11); P, 5.59 (5.69) %. 31P-NMR (CD2Cl2): δ=139.2 (s) ppm. 1H-NMR (CD2Cl2): 1.29 (s, 9H, 3CH3), 6.65-6.71 (m, 2H), 7.05-7.15 (m, 9H), 7.19-7.29 (m, 9H), 7.56-7.61 (m, 4H). 13C-NMR (CD2Cl2): δ=31.2 (3CH3), 34.2 (C), 95.2 (d, JCP=7.73 Hz), 120.1 (d, JCP=7.73 Hz), 126.5, 127.2 (d, JCP=18.9 Hz), 127.3 (d, JCP=18.9 Hz), 128.8 (d, JCP=3.54 Hz), 129.9, 142.6 (d, JCP=4.02 Hz), 142.7, 146.9, 148.6 (d, JCP=8.68 Hz) ppm.

### 2-(2,4-di-tert-butylphenoxy)-4,4,5,5-tetraphenyl-1,3,2-dioxaphospholane

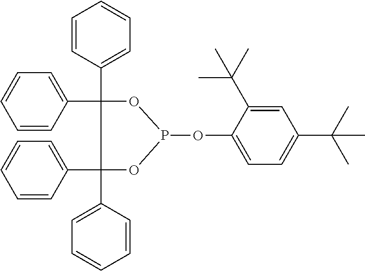 [SMILES: CC(C)(C)c1ccc(OP2OC(c3ccccc3)(c3ccccc3)C(c3ccccc3)(c3ccccc3)O2)c(C(C)(C)C)c1]

To a stirred suspension of 2,4-di-tert-butylphenol (0.247 g, 1.201 mmol) in Toluene (5 ml), Triethylamine (0.301 g, 2.97 mmol, 4 mL) was added dropwise at 0° C. The reaction mixture was treated dropwise with cold solutions of 2-Chloro-4,4,5,5-tetraphenyl-1,3,2-dioxaphospholan (0.517 g; 1.201 mmol) in Toluene (5 ml). The reaction mixture was stirred 24 hrs at room temperature and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.395 g (0.66 mmol, 55%). Elemental analysis calc. for C40H41O3P=600.72 g/mol): C, 79.94 (79.97); H, 6.89 (6.88); P, 5.26 (5.16) %. 31P-NMR (CD2Cl2): 8=138.5 (s) ppm. 1H-NMR (CD2Cl2): 1.25 (s, 9H, 3CH3), 1.32 (s, 9H, 3CH3), 6.90 (d, J=8.18 Hz) 7.05-7.20 (m, 17H), 7.36 (d, J=2.24 Hz, 1H), 7.56-7.59 (m, 4H). 13C-NMR (CD2Cl2): δ=30.4 (3CH3), 31.8 (3CH3), 34.9 (C), 35.1 (C), 95.8 (d, JCP=8.23 Hz), 121.7 (d, JCP=15.3 Hz), 124.0, 124.8, 127.6, 127.7, 127.8, 129.3 (d, JCP=3.38 Hz), 130.3, 140.7 (d, JCP=2.57 Hz), 142.6 (d, J=4.31 Hz), 143.2, 146.9, 148.4 (d, JCP=10.0 Hz) ppm. ESI-TOF/HRMS calc. For for C40H41O3P (M+H)+; 601.2866 found; 601.2866.

### 2-(2,6-di-tert-butylphenoxy)-4,4,5,5-tetraphenyl-1,3,2-dioxaphospholane

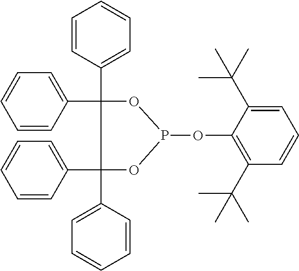 [SMILES: CC(C)(C)c1cccc(C(C)(C)C)c1OP1OC(c2ccccc2)(c2ccccc2)C(c2ccccc2)(c2ccccc2)O1]

To a stirred suspension of 2,6-di-tert-butylphenol (0.412 g, 2.00 mmol) in THF (5 ml), n-BuLi (1.00 mL, 2.50 mmol) 2.5 M in hexane was added dropwise at 0° C., mixture was stirred for 20 min., and warmed gently to room temperature. Then it was treated dropwise with cold solutions of 2-Chloro-4,4,5,5-tetraphenyl-1,3,2-dioxaphospholan (0.861 g, 2.00 mmol) in THF (5 ml). The reaction mixture was stirred 24 hrs at room temperature and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h.

Yield: 0.853 g (1.42 mmol, 71%). 31P-NMR (CD2Cl2): δ=145.6 (s) ppm. 1H-NMR (CD2Cl2): 1.36 (s, 18H, 9CH3), 6.97-7.22 (m, 18H), 7.32 (d, J=8.31 Hz, 2H), 7.62-7.67 (m, 3H). 13C-NMR (CD2Cl2): δ=31.9 (3CH3), 32.0 (3CH3), 35.5 (C), 96.2 (d, JCP=8.98 Hz), 124.2, 126.7, 127.6 (d, JCP=23.6 Hz), 127.7 (d, JCP=34.4 Hz), 129.2 (d, JCP=4.27 Hz), 130.8, 142.4 (d, JCP=4.75 Hz), 142.7, 144.3 (d, J=3.21 Hz), 158.8 (d, JCP=11.8 Hz) ppm. ESI-TOF/HRMS calc. For for C40H41O3P (M+H)+; 601.2856 found; 601.2866.

### 4,4,5,5-tetraphenyl-2-(2,4,6-tri-tert-butylphenoxy)-1,3,2-dioxaphospholane

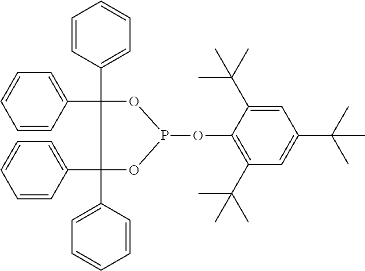 [SMILES: CC(C)(C)c1cc(C(C)(C)C)c(OP2OC(c3ccccc3)(c3ccccc3)C(c3ccccc3)(c3ccccc3)O2)c(C(C)(C)C)c1]

To a stirred suspension of 2,4,6-tri-tert-butylphenol (0.393 g, 1.50 mmol) in THF (5 ml), n-BuLi (0.60 mL, 1.00 mmol) 2.5 M in hexane was added dropwise at 0° C., mixture was stirred for 20 min., and warmed gently to room temperature. Then it was treated dropwise with cold solutions of 2-Chloro-4,4,5,5-tetraphenyl-1,3,2-dioxaphospholan (0.646 g, 1.50 mmol) in THF (5 ml). The reaction mixture was stirred 24 hrs at room temperature and filtered. The filtrate was evaporated to dryness in vacuo. The residue obtained was dissolved in toluene (15 ml), the solution was filtered through a thin layer of silica and the solvent removed in vacuo and the solid product dried at 0.1 bar, 40° C., for 5 h. Yield: 0.541 g (0.82 mmol, 55%). Elemental analysis calc. for C44H49O3P=656.8 g/mol): C, 80.38 (80.46); H, 7.38 (7.52); P, 4.78 (4.72) %. 31P-NMR (CD2Cl2): δ=145.7 (s) ppm. 1H-NMR (CD2Cl2): 1.34 (s, 9H, 3CH3), 1.39 (s, 18H, 6CH3), 7.02-7.23 (m, 16 Hz) 7.37 (s, 1H), 7.63-7.69 (m, 4H). 13C-NMR (CD2Cl2): δ=31.8 (3CH3), 32.0 (3CH3), 32.2 (2CH3), 32.5 (C), 35.2 (C), 35.8 (C), 96.2 (d, JCP=8.88 Hz), 123.8, 127.5, (d, JCP=22.8 Hz), 127.9, 129.3 (d, JCP=3.90 Hz), 142.5 (d, JCP=4.80 Hz), 142.8, 143.1 (d, JCP=3.90 Hz), 146.0, 148.3 (d, JCP=11.5 Hz) ppm. ESI-TOF/HRMS calc. for C44H49O3P (M+H)+; 657.3492 found; 657.3492.

Numerous modifications and variations on the present invention are possible in light of the above teachings. It is therefore to be understood that within the scope of the appended claims, the invention may be practiced otherwise than as specifically described herein.

1. A monophosphite compound selected from the group of structures consisting of structures (I) to (V): 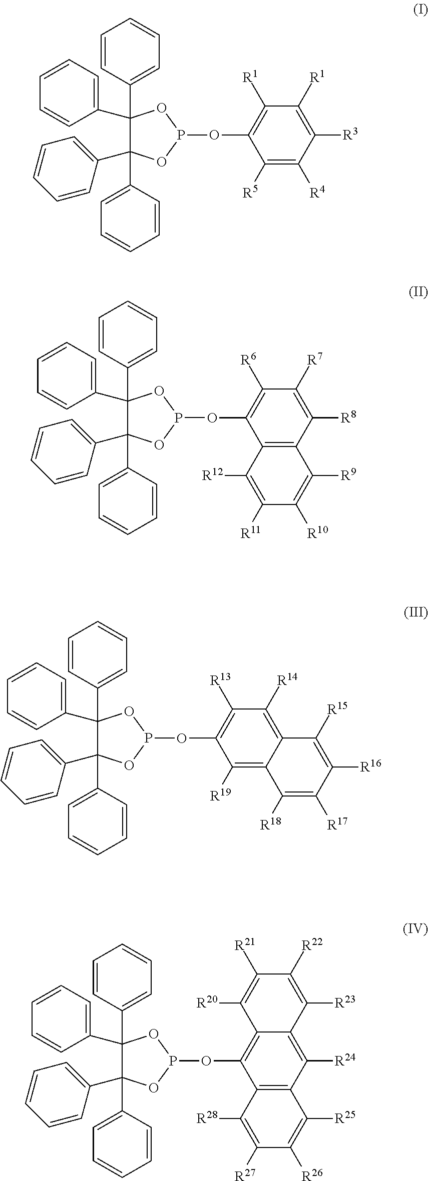 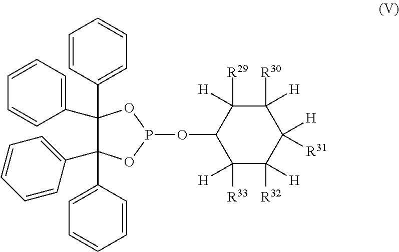
wherein
R¹, R², R³, R⁴, R⁵ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂ or —N[(C₁-C₁₂)-alkyl]₂;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;
R²⁹, R³⁰, R³¹, R³², R³³ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;
wherein when any of R¹ to R³³ comprises any of an alkyl and an aryl group, the alkyl group and the aryl group may optionally be substituted,
and with the proviso that R¹ and R⁵ are not tert-butyl, at least one of the R¹, R², R³, R⁴, R⁵ is not —H, and if one of R¹, R², R³, R⁴, R⁵ is phenyl, at least one of the four remaining R groups is not —H.

2. The monophosphite compound of claim 1,
wherein R¹, R², R³, R⁴, R⁵ are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

3. The monophosphite compound of claim 1,
wherein R⁶, R⁷, R⁸, R⁹, R¹⁰, R¹¹, R¹² are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

4. The monophosphite compound of claim 1,
wherein R¹³, R¹⁴, R¹⁵, R¹⁶, R¹⁷, R¹⁸, R¹⁹ are each independently —H, —(C₁-C₁₂)-alkyl, —O—(C₆-C₂₀)-aryl, —(C₆-C₂₀)-aryl, —S-alkyl or —S-aryl.

5. The monophosphite compound of claim 1,
wherein R²⁰, R²¹, R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl or —S-aryl.

6. The monophosphite compound of claim 1,
wherein R²⁹, R³⁰, R³¹, R³², R³³ are each independently —H, —O—(C₁-C₁₂)-alkyl or —O—(C₆-C₂₀)-aryl.

7. The monophosphite compound of claim 1,
wherein R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —O—(C₁-C₁₂)-alkyl, —O—(C₆-C₂₀)-aryl, —(C₆-C₂₀)-aryl, —S-alkyl or —S-aryl.

8. The monophosphite compound of claim 1, wherein the compound is of structure (I)

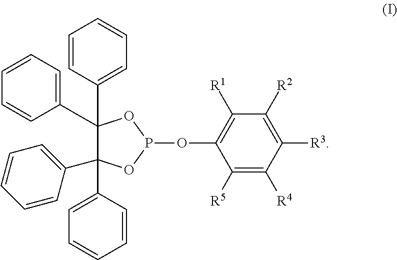

9. The monophosphite compound of claim 1,
wherein the compound is of structure (II):

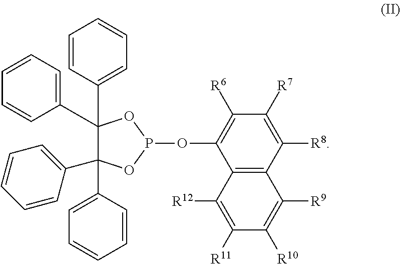

10. The monophosphite compound of claim 1,
wherein the compound is of structure (III):

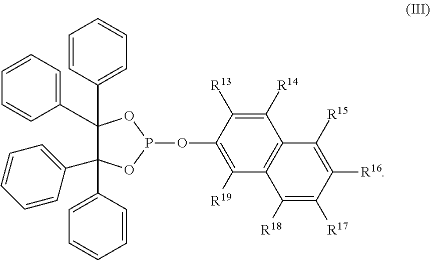

11. The monophosphite compound of claim 1,
wherein the compound is of structure (IV):

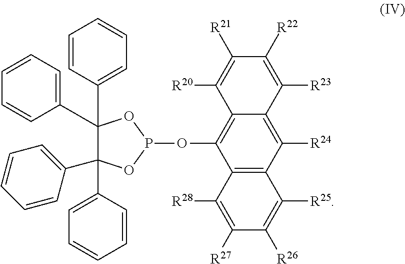

12. The monophosphite compound of claim 1,
wherein the compound is of structure (V):

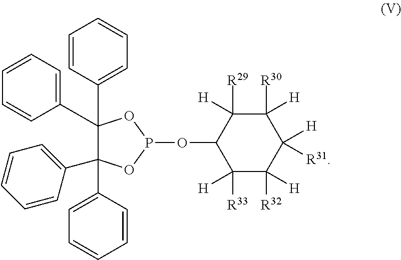

13. A metal ligand complex comprising:
the monophosphite compound of claim 1; and
a metal atom selected from the group consisting of Rh, Ru, Co and Ir.

14. A method for hydroformylation comprising:
conducting the hydroformylation reaction in the presence of the metal ligand complex of claim 13.

15. The method for hydroformylation of claim 14, further comprising:
a) charging an olefin to a reaction device;
b) adding a catalyst comprising the metal ligand complex to the reaction device;
or adding a catalyst comprising a monophosphite compound selected from the group of structures consisting of structures (I) to (V): 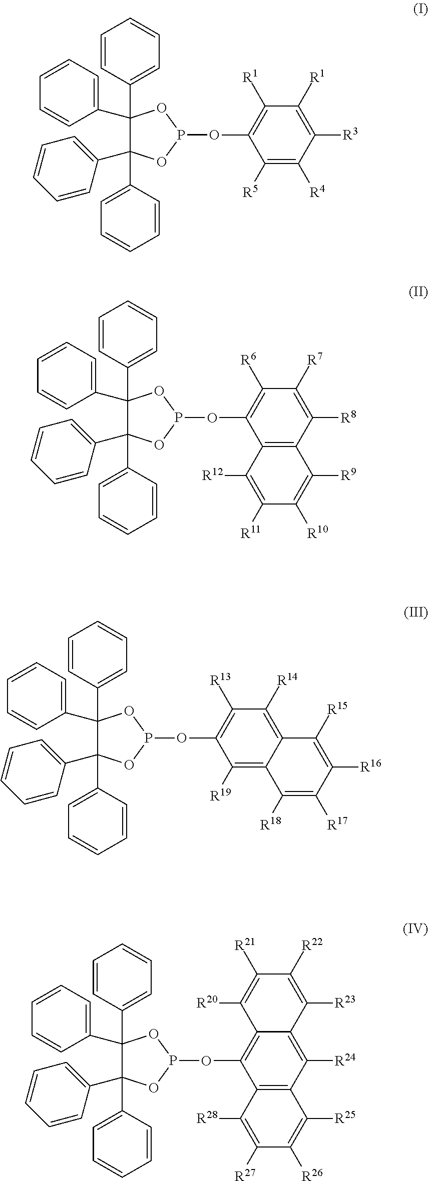 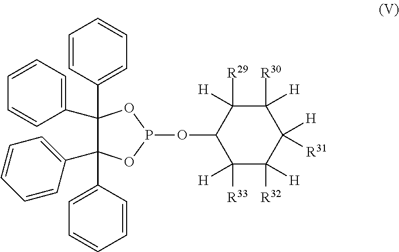
and a substance having a metal atom selected from the group consisting of Rh, Ru, Co and Ir;
c) feeding H₂ and CO into the reaction device to the olefin and catalyst to obtain a reaction mixture; and
d) heating the reaction mixture to effect conversion of the olefin to an aldehyde;
wherein
R¹, R², R³, R⁴, R⁵ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —CN, —NH₂ or —N[(C₁-C₁₂)-alkyl]₂;
R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ R²², R²³, R²⁴, R²⁵, R²⁶, R²⁷, R²⁸ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;
R²⁹, R³⁰, R³¹, R³², R³³ are each independently —H, —(C₁-C₁₂)-alkyl, —O—(C₁-C₁₂)-alkyl, —O—(C₆-C₂₀)-aryl, —(C₆-C₂₀)-aryl, —S-alkyl, —S-aryl, halogen, —COO—(C₁-C₁₂)-alkyl, —CONH—(C₁-C₁₂)-alkyl, —CO—(C₁-C₁₂)-alkyl, —CO—(C₆-C₂₀)-aryl, —COOH, —OH, —SO₃H, —NH₂ or —N[(C₁-C₁₂)-alkyl]₂;
wherein when any of R¹ to R³³ comprises any of an alkyl and an aryl group, the alkyl group and the aryl group may optionally be substituted,
and with the proviso that R¹ and R⁵ are not tert-butyl, at least one of the R¹, R², R³, R⁴, R⁵ is not —H, and if one of R¹, R², R³, R⁴, R⁵ is phenyl, at least one of the four remaining R groups is not —H.

16. The method for hydroformylation of claim 15, wherein the olefin is a monoolefin having 2 to 24 carbon atoms.

17. The method for hydroformylation of claim 15, wherein the monoolefin is a terminal olefin or an internal olefin.

18. The method for hydroformylation of claim 15, wherein the catalyst comprises the monophosphite compound not complexed to a metal.

19. The method for hydroformylation of claim 15, wherein the metal is Rh.
